(19) United States
(12) Patent Application Publication
(10) Pub. No.: US 2023/0016312 A1
(43) Pub. Date: Jan. 19, 2023

(54) ANEURYSM TREATMENT WITH PUSHABLE IMPLANTED BRAID

(71) Applicant: DePuy Synthes Products, Inc., Raynham, MA (US)

(72) Inventors: Ruijiao XU, Miami Lakes, FL (US); Lacey GOROCHOW, Miami, FL (US)

(73) Assignee: DePuy Synthes Products, Inc., Raynham, MA (US)

(21) Appl. No.: 17/952,505

(22) Filed: Sep. 26, 2022

Related U.S. Application Data
(62) Division of application No. 16/853,135, filed on Apr. 20, 2020, now Pat. No. 11,497,504.

(51) Int. Cl. A61B 17/12

(52) U.S. Cl. CPC A61B 17/12113; A61B 17/12145

(57) ABSTRACT

The present invention provides a braided implant with a retractable dual proximal layer and methods for administering the braided implant to treat aneurysms. The implant can include a tubular braid that can be set into a predetermined shape, compressed for delivery through a microcatheter, and implanted in at least one implanted position that is based on the predetermined shape and the geometry of the aneurysm in which the braid is implanted. The implant can also have a retractable dual layer at the proximal end of the device made of the same braid to provide additional coverage at the neck of the aneurysm. The dual layer can be pressed distally into a first implanted portion of the tubular braid, moving the first portion of the tubular braid towards the distal portion of an aneurysm wall so that the implant can partially or completely occlude an aneurysm neck.

### CROSS-REFERENCE TO RELATED APPLICATIONS

This application is a Divisional of U.S. application Ser. No. 16/853,135 filed Apr. 20, 2020, which is a Continuation-In-Part to U.S. application Ser. No. 16/418,199 filed May 21, 2019, now U.S. Pat. No. 10,653,425, both of which are incorporated herein in their entirety.

### FIELD OF THE INVENTION

The present invention generally relates to medical instruments, and more particularly, to embolic implants for aneurysm therapy.

### BACKGROUND

Aneurysms can be complicated and difficult to treat. For example, treatment access may be limited or unavailable when an aneurysm is located proximate critical tissues. Such factors are of particular concern with cranial aneurysms due to the brain tissue surrounding cranial vessels and the corresponding limited treatment access.

Prior solutions have included endovascular treatment access whereby an internal volume of the aneurysm sac is removed or excluded from arterial blood pressure and flow. In this respect, because the interior walls of the aneurysm may continue being subjected to flow of blood and related pressure, aneurysm rupture remains possible.

Alternatives to endovascular or other surgical approaches can include occlusive devices. Such devices have typically incorporated multiple embolic coils that are delivered to the vasculature using microcatheter delivery systems. For example, when treating cranial aneurysms, a delivery catheter with embolic coils is typically first inserted into non-cranial vasculature through a femoral artery in the hip or groin area. Thereafter, the catheter is guided to a location of interest within the cranium. The sac of the aneurysm can then be filled with the embolic material to create a thrombotic mass that protects the arterial walls from blood flow and related pressure. However, such occlusive devices do have certain shortcomings, including mass effect, which can cause compression on the brain and its nerves.

For example, embolic coils delivered to the neck of the aneurysm can potentially have the adverse effect of impeding the flow of blood in the adjoining blood vessel, particularly if the entrance is overpacked. Conversely, if the entrance is insufficiently packed, blood flow can persist into the aneurysm. Treating certain aneurysm morphology (e.g., wide neck, bifurcation, etc.) can require ancillary devices such a stents or balloons to support the coil mass and obtain the desired packing density. Once implanted, the coils cannot easily be retracted or repositioned. Furthermore, embolic coils do not always effectively treat aneurysms as aneurysms treated with multiple coils often recanalize or compact because of poor coiling, lack of coverage across the aneurysm neck, blood flow, or large aneurysm size.

Another particular type of occlusive approach endeavors to deliver and treat the entrance or “neck” of the aneurysm. In such “neck” approaches, by minimizing blood flow across the neck, a cessation of flow into the aneurysm may be achieved. It is understood that the neck plane is an imaginary surface where the inner most layer of the parent wall would be but for the aneurysm. However, neck-occlusive approaches, such as implanting a flow impeding device in the parent vessel, are not without drawbacks. Such an approach may impede blood flow into peripheral blood vessels while blocking the aneurysm neck in the parent vessel. Impeding flow to the peripheral blood vessel can unintentionally lead to severe damage if the openings of the vessels are blocked.

Alternatives to embolic coils are being explored, such as tubular braided implants. Tubular braided implants have the potential to easily, accurately, and safely treat an aneurysm or other arterio-venous malformation in a parent vessel without blocking flow into perforator vessels communicating with the parent vessel. Compared to embolic coils, however, tubular braided implants are a newer technology, and there is therefore capacity for improved geometries, configurations, delivery systems, etc. for the tubular braided implants. For instance, delivery of tubular braided implants can require unique delivery systems to prevent the braid from inverting or abrading when pushed through a microcatheter, and some simple delivery systems that push embolic coils through microcatheters from their proximal end may not be effective to deliver tubular braids.

There is therefore a need for improved methods, devices, and systems for implants for aneurysm treatment.

### SUMMARY

It is an object of the present invention to provide systems, devices, and methods to meet the above-stated needs. Generally, it is an object of the present invention to provide a braided implant with a retractable dual proximal layer. The implant can secure within an aneurysm sac and occlude at least a majority of the aneurysm's neck. The implant can include a tubular braid that can be set into a predetermined shape, compressed for delivery through a microcatheter, and implanted in at least one implanted position that is based on the predetermined shape and the geometry of the aneurysm in which the braid is implanted. The implant can also have a retractable dual layer at the proximal end of the device made of the same braid to provide additional coverage at the neck of the aneurysm.

In some examples presented herein, the dual layer can be shaped by expanding it radially, and the dual layer can be pressed distally into a first portion of the tubular braid already within the aneurysm. By pressing the dual layer distally into the first portion of the tubular braid, the first portion of the tubular braid can be moved towards the distal portion of an aneurysm wall so that the implant can partially or completely occlude an aneurysm neck. Pushing the dual layer into the first portion of the braid can help conform the implant to the shape of the aneurysm and resist compaction. The dual layer, when expanded radially and pressed into the first portion of the braid, also can provide additional coverage at the neck of the aneurysm to increase thrombosis. In some examples, the dual layer can also be placed within the aneurysm sac with only a detachment point external to the sac.

In some examples, the tubular braid can include memory shape material that can be heat set to a predetermined shape, can be deformed for delivery through a catheter, and can self-expand to an implanted shape that is based on the predetermined shape and confined by the anatomy of the aneurysm in which it is implanted.

In some examples the tubular braid can be shaped to a delivery shape that is extended to a single layer of tubular braid having a compressed circumference/diameter sized to be delivered through the microcatheter.

In some examples, before the implant is released from the delivery system, the implant can be partially or fully retracted into the microcatheter and repositioned.

An example method for forming an occlusive device to treat an aneurysm can include one or more of the following steps presented in no particular order, and the method can include additional steps not included here. An implant with a tubular braid, an open end, and a pinched end can be selected. The tubular braid can be shaped to a predetermined shape. Shaping the tubular braid to a predetermined shape can also include further steps. These steps can include inverting the tubular braid to form a distal inversion. The tubular braid can also be inverted to form a proximal inversion by moving the open end over at least a portion of the braid. A first segment of the tubular braid extending between the open end and the proximal inversion can be shaped. A second segment of the tubular braid extending between the proximal inversion and the distal inversion can be shaped. The open end can be positioned to encircle the second segment. A third segment extending from the distal inversion to the proximal inversion can be shaped. The second segment can be positioned to surround the third segment. A fourth segment of the tubular braid extending from the third segment radially outward from a central axis to cross the proximal inversion can be shaped and can fold and converge at the pinched end. The fourth segment can be positioned near the neck of an aneurysm.

An example method for treating an aneurysm can include one or more of the following steps presented in no particular order, and the method can include additional steps not included here. A first portion of a tubular braided implant, which can have a tubular braid, an open end, and a pinched end, can be positioned within a sac of the aneurysm such that the first portion circumferentially apposes walls within the sac. The first portion can have one or more inversions. A second portion of the tubular braided implant can be expanded radially to occlude a majority of a neck of the aneurysm. The second portion can be pressed distally into the first portion. The first portion of the tubular braided implant can be moved toward a distal portion of the aneurysm wall as a result of pressing the second portion distally into the first portion.

In some examples, expanding the second portion of the tubular braided implant can include positioning a fold in the second segment to define a substantially circular perimeter of the second portion and compressing the second portion along a central axis of the tubular braided implant such that the second portion can have a substantially circular shape having an area and two layers of braid over a majority of the area of the substantially circular shape.

In some examples, positioning the first portion of the tubular braided implant can further include shaping the tubular braided implant to form a columnar post encircling a central axis of the tubular braided implant and extending a majority of a height of the first portion. In another example, positioning the first portion of the tubular braided implant can further involve positioning a proximal inversion near the neck of the aneurysm and positioning a distal inversion approximate the distal portion of the aneurysm wall. In another example, positioning the first portion of the tubular braided implant can further involve positioning the open end of the tubular braided implant to circumferentially appose the aneurysm wall, shaping a first segment of the tubular braid extending between the open end and the proximal inversion to appose at least a portion of a wall of the aneurysm within the aneurysm's sac, and shaping a second segment of the tubular braid such that the first segment provides an outwardly radial force in a plane defining a boundary between the aneurysm and blood vessel branches, the force sufficient to appose the first segment to walls of the aneurysm.

In some examples, pressing the second portion distally into the first portion can further involve pressing the second portion of the tubular braided implant against the proximal inversion in the first portion of the tubular braided implant.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A is an illustration of an example implant having a tubular braid in a predetermined shape according to aspects of the present invention;

FIG. 1B is an illustration of the example implant with the tubular braid in a first implanted shape according to aspects of the present invention;

FIGS. 2A through 2I are illustrations of an implant having a tubular braid that expands to an implanted shape similar to as illustrated in FIG. 1B as the tubular braid exits a microcatheter according to aspects of the present inventions;

FIGS. 3A through 3B are flow diagrams for a method of forming an occlusive device to treat an aneurysm; and

FIGS. 4A through 4B are flow diagrams for a method for treating an aneurysm.

### DETAILED DESCRIPTION

Examples presented herein generally include a braided implant that can secure within an aneurysm sac and occlude a majority of the aneurysm's neck. The implant can include a tubular braid that can be set into a predetermined shape, compressed for delivery through a microcatheter, and implanted in at least one implanted position that is based on the predetermined shape and the geometry of the aneurysm in which the braid is implanted. The implant can include a single layer of braid (e.g., a braid that can be extended to form a single layer tube) heat treated into multiple layers with retractable dual layer at the proximal end of the tubular braid. When compressed, the implant can be sufficiently short to mitigate friction forces produced when the implant is delivered unsheathed through the microcatheter.

A first portion of the tubular braid can be positioned in an aneurysm, after which the retractable dual layer can be deployed from the microcatheter and pushed onto the first portion of the tubular braid. This configuration provides three layers of braid at the neck of the aneurysm. The dual layer can potentially cover any gap between the first portion of implanted tubular braid and the aneurysm neck, and can potentially increase metal coverage, decrease porosity of the implant, and increase stasis and blood flow diversion at the neck of the aneurysm to promote the sealing and healing of the aneurysm compared a similarly shaped braided implant lacking the dual layer. The entire implant can be retractable until a desired position is achieved.

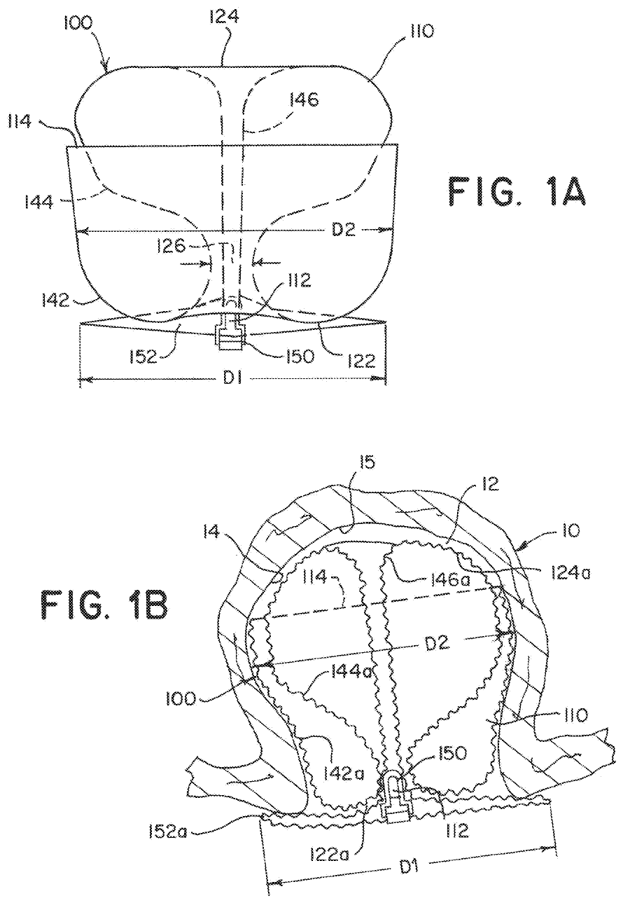

FIGS. 1A and 1B are illustrations of an example braided implant 100 that can have a predetermined shape as shown in FIG. 1A and a distinct implanted shape as illustrated in FIG. 1B. The implant 100 can treat a range of aneurysm sizes. The implant 100 can include a tubular braid 110 having an open end 114 and a pinched end 112. The implant 100 can include a connection and detachment feature 150 (referred to equivalently as “connection feature” and “detachment feature” herein) attached to the braid 110 at the pinched end 112. The pinched end 112 can include a marker band and/or soldered point with visibility, and/or the connection feature 150 can include radiopaque material. The tubular braid 110 can be formed in the predetermined shape (FIG. 1A), collapsed for delivery through a microcatheter, attached to a delivery system at connection feature 150, and implanted in an implanted shape such as the one shown in FIG. 1B.

Referring to FIG. 1A, when in the predetermined shape, the tubular braid 110 can include two inversions 122, 124, a pinched end 112, and an open end 114. The tubular braid 110 can include four segments, 142, 144, 146, and 152. The first segment 142 can extend from the open end 114 of the tubular braid 110 to a proximal inversion 122. The second segment 144 can be encircled by the open end 114 and extend from the proximal inversion 122 to a distal inversion 124. The third segment 146 can be surrounded by the second segment 144 and extend from the distal inversion 124 to the proximal inversion 122. The first segment 142, second segment 144, and third segment 146 can form the first portion of the tubular braid 110. The fourth segment 152 can extend from the third segment 146 radially outward from a central axis to cross the proximal inversion 122, fold, and converge at the pinched end 112. The fourth segment 152 can be partially encircled by the proximal inversion 122.

When in the predetermined shape, the tubular braid 110 can be substantially radially symmetrical about a central vertical axis. The detachment feature 150 is illustrated in FIG. 1A as a flat key that can be used with a mechanical delivery implant system (not pictured). The tubular braid 110 can be formed into the predetermined shape by first inverting the braid outwardly to separate the third segment 146 from the second segment 144 with a distal inversion 124. Then, the second segment 144 can be shaped over a form to produce the substantially “S” shaped profile illustrated in FIG. 1A. Next, the braid 110 can be inverted outwardly again to separate the second segment 144 from the first segment 142 with a proximal inversion 122. Finally, the fourth segment 152 can be shaped by expanding the fourth segment 152 radially. The fourth segment 152 can be pressed distally into the first portion of the tubular braid 110. It can also be advantageous to minimize a neck opening 126 defined by the lower extension of the “S” shape of second segment 144 to maximize occlusion of an aneurysm neck when the implant 100 is implanted.

The tubular braid 110 can include memory shape material that can be heat set to a predetermined shape, can be deformed for delivery through a catheter, and can self-expand to an implanted shape that is based on the predetermined shape and confined by the anatomy of the aneurysm in which it is implanted. When the tubular braid 110 is in the predetermined shape as depicted in FIG. 1A, the fourth segment 152 can comprise a diameter D1 greater than or approximately equal to a maximum diameter D2 of the first segment 142. Alternatively, when the tubular braid 110 is in the predetermined shape as depicted in FIG. 1A, the fourth segment 152 can comprise a diameter D1 lesser than a maximum diameter D2 of the first segment 142. When the tubular braid 110 is in the predetermined shape (FIG. 1A), the second segment 144 can form a sack, and at least a portion of the third segment 146 can positioned within the sack and at least a portion of the fourth segment 152 can be positioned external to the sack. As illustrated (FIG. 1B), when implanted, the fourth segment 152 can be positioned external to the aneurysm sac, extending across the aneurysm neck 16. Preferably, the fourth segment 152 can appose vasculature walls surrounding the aneurysm neck 16 when implanted. Alternatively, the shaped fourth segment 152 can also be placed within the aneurysm sac. The detachment feature 150 can be implanted centrally in the aneurysm neck 16. The detachment feature 150 can be positioned external to the sac 12.

The tubular braid 110 in the implanted shape (FIG. 1B) can be radially or vertically compressed or extended compared to the predetermined shape. As illustrated in FIG. 1B, when in the implanted shape, the braid 110 can have an outer layer 142a corresponding to the first segment 142 of the predetermined shape and positioned to contact an aneurysm wall 14 of the aneurysm 10, a proximal inversion 122a corresponding to the proximal inversion 122 of the predetermined shape and positioned to be placed approximate a neck 16 of the aneurysm 10, and a sack 144a corresponding to the second segment 144 of the predetermined shape and positioned to appose a portion of the aneurysm wall 14 of the aneurysm 10 and apposing the outer layer 142a. A distal inversion 124a can correspond to the distal inversion 124 of the predetermined shape, a third segment 146a can correspond to the third segment 146 in the predetermined shape. The braid 110 can also have a fourth segment 152a corresponding to the fourth segment 152 of the predetermined shape and extending from the third segment 146a radially outward from a central axis to cross the proximal inversion 122a, fold, and converge at the pinched end 112. As described in FIG. 1A, the fourth segment 152a can be pressed distally into the first portion of the tubular braid 110.

By pressing the fourth segment 152a distally into the first portion of the tubular braid 110, the first portion 142a, 144a, 146a of the tubular braid 110 can be moved towards the distal portion of an aneurysm wall 15 to occlude a portion of the neck 16 of the aneurysm 10. Pushing the fourth segment 152a into the first portion of the braid 110 can help conform the implant 100 to the shape of the aneurysm 10 and resist compaction. The fourth segment 152a when expanded radially and pressed into the first portion of the braid 110 also can provide additional coverage at the neck 16 of the aneurysm 10 to increase thrombosis and seal the aneurysm 10. When the fourth segment 152a is pressed into the first portion of the braid 110, three layers of braid are present at the neck of the aneurysm. The fourth segment 152a can cover spatial gaps between the first portion of implanted tubular braid 110 and the aneurysm neck 16, and can potentially increase metal coverage, decrease porosity of the implant 100, and increase stasis and blood flow diversion at the neck 16 of the aneurysm 10 to promote the sealing and thrombosis of the aneurysm 10. The fourth segment 152a can be shaped to occlude the majority of an aneurysm neck 16 when the device 100 is implanted. The fourth segment 152a can be shaped to completely occlude an aneurysm neck 16 when the device 100 is implanted.

When the tubular braid 110 is in the implanted shape (FIG. 1B), the fourth segment 152a can comprise a diameter D1 greater than or approximately equal to a maximum diameter D2 of the first segment 142a. Alternatively, when the tubular braid 110 is in the implanted shape (FIG. 1B), the fourth segment 152a can comprise a diameter D1 lesser than a maximum diameter D2 of the first segment 142a. When the tubular braid 110 is in the implanted shape (FIG. 1B), the second segment 144a can form a sack, and at least a portion of the third segment 146a can be positioned within the sack and at least a portion of the fourth segment 152a can be positioned external to the sack. The shaped fourth segment 152a can also be placed within the aneurysm sac 12 with only the detachment point 150 external to the sac 12.

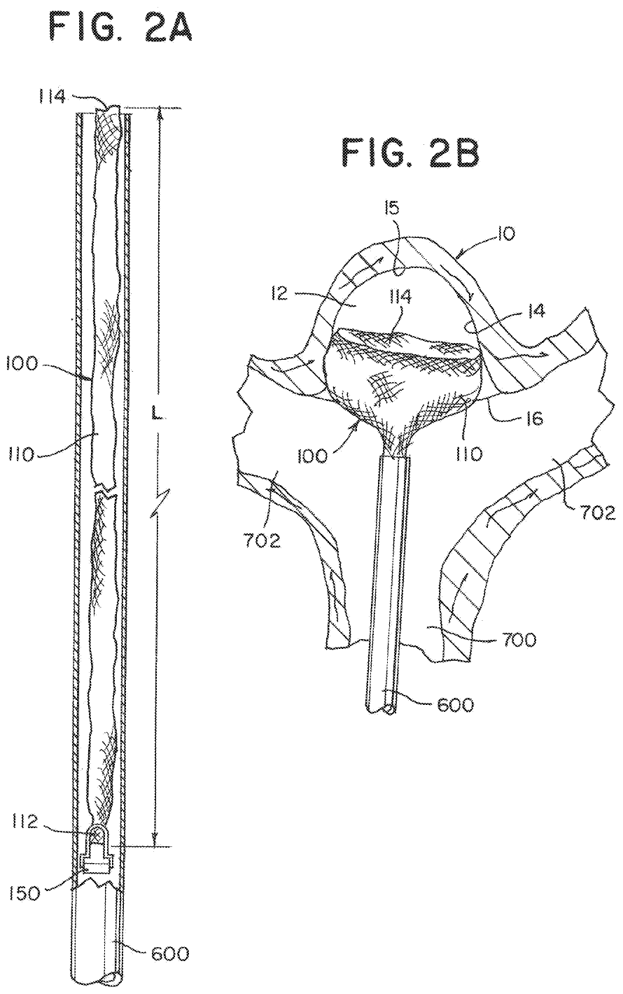

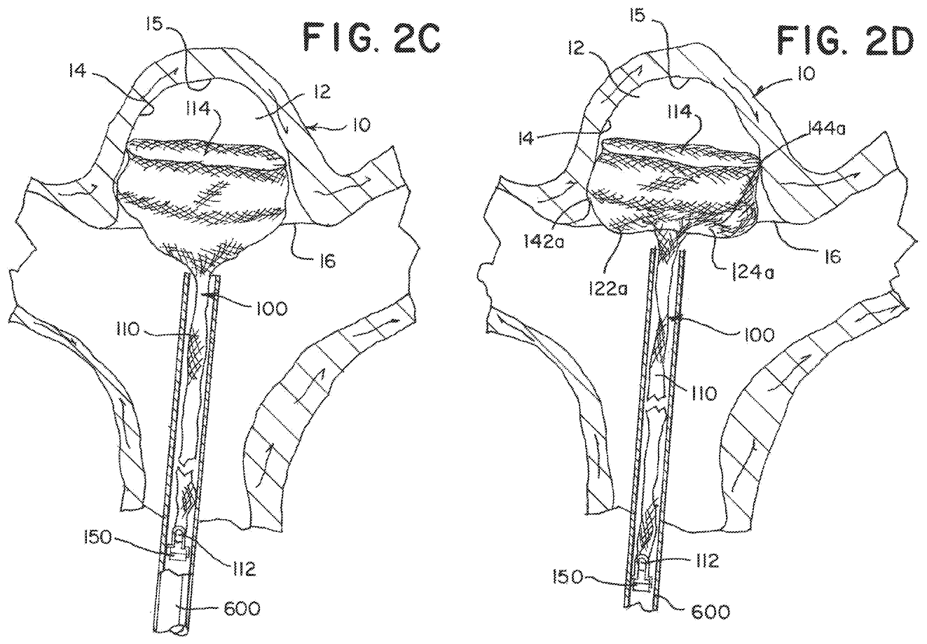

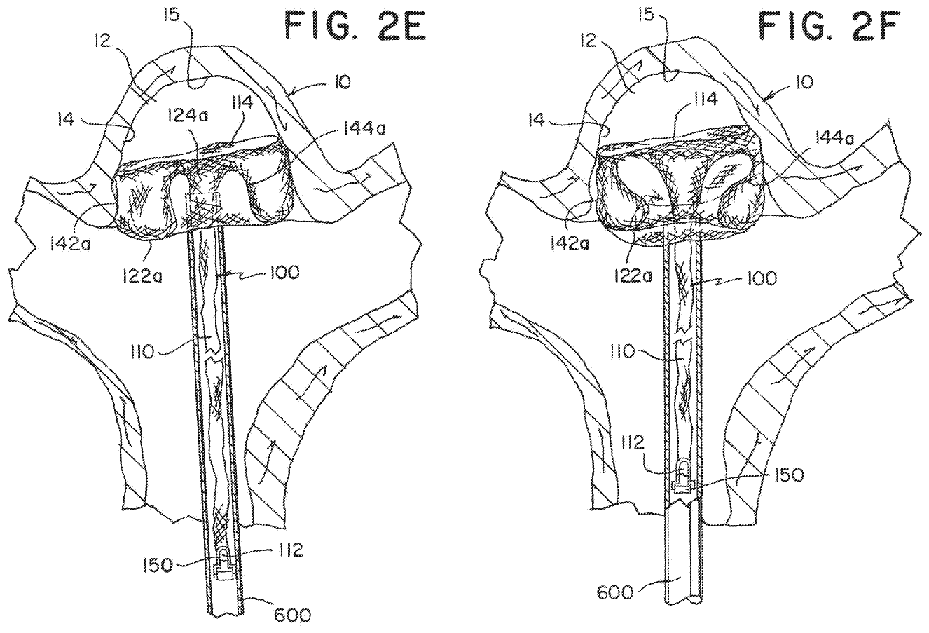

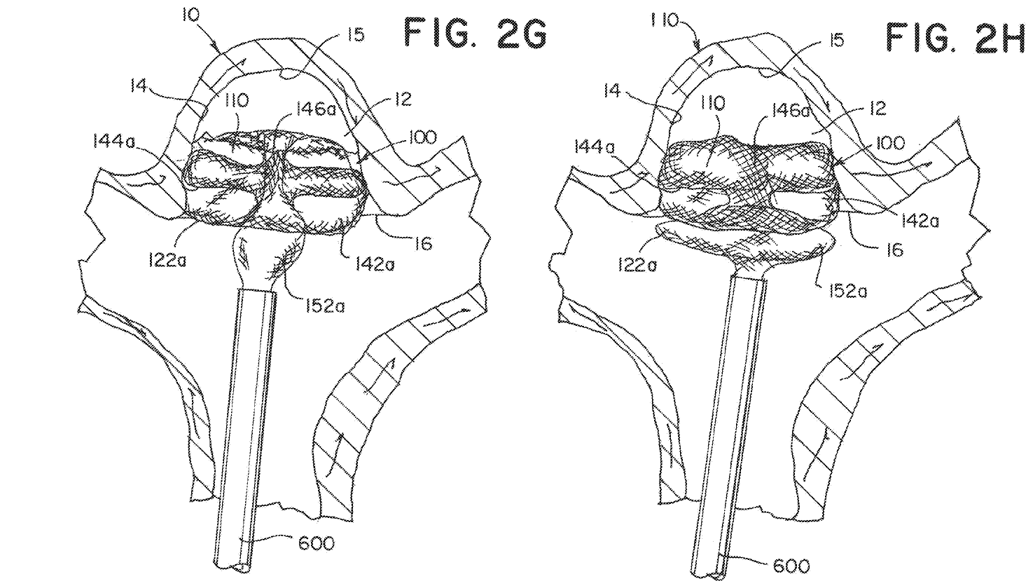

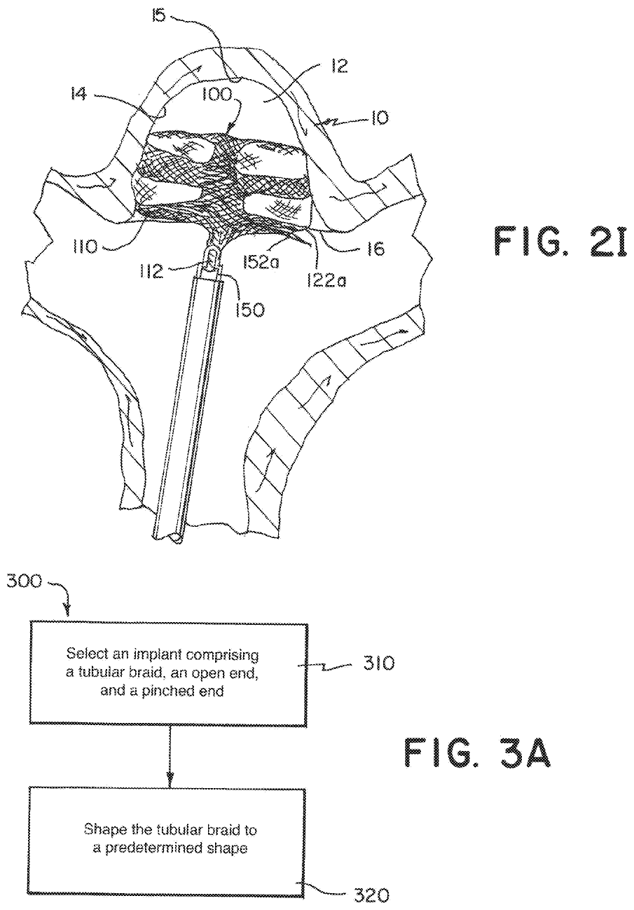

FIGS. 2A through 2I are illustrations of an example implant 100 having a braid 110 expanding to an implanted shape that is based on a predetermined shape and the anatomy of the aneurysm and nearby blood vessel as the braid 110 exits a microcatheter 600. The implant 100 has a predetermined shape similar to the shape illustrated in FIG. 1A. As illustrated in FIG. 2A, the braid 110 can be shaped to a delivery shape that is extended to a single layer of tubular braid having a compressed circumference/diameter sized to be delivered through the microcatheter 600 and a length L. As will be appreciated and understood by a person of ordinary skill in the art, the length L of a specific braid 110 can be tailored based on the size and shape of the aneurysm being treated. The length L can be approximately 1 inch in length.

During delivery through the microcatheter 600, the detachment feature 150 can be attached to a delivery system at a proximal end of the implant 100, the pinched end 112 can be positioned near the proximal end of the implant 100, and the open end 114 can define the distal end of the implant 100. Collapsing the braid 110 to a single layer tube can result in a braid 110 that has a sufficiently small diameter and a sufficiently short length L to mitigate effects of friction force on the braid 110 when it is delivered through the microcatheter, allowing the braid 110 to be delivered unsheathed in some applications

As illustrated in FIG. 2B, the implant 100 can be delivered to an aneurysm 10 through the microcatheter 600. The open end 114 can be positioned to exit the microcatheter 600 before any other portion of the braid 110 exits the microcatheter. The open end 114 can expand within the aneurysm sac 12 as it exits the microcatheter 600. The illustrated aneurysm 10 is positioned at a bifurcation including a stem blood vessel 700 and two branch vessels 702, and the microcatheter 600 is illustrated being delivered through the stem blood vessel 700. It is contemplated that the implant could be delivered to an aneurysm on a sidewall of a blood vessel through a curved microcatheter, and such a procedure is intended to be embraced by the scope of the present disclosure. As illustrated in FIG. 2C, the distal portion of the braid 110 can continue to expand radially within the aneurysm sac 12 as it exits the microcatheter 600. As the braid 110 is further pushed distally from the microcatheter 600, the braid 110 can appose the aneurysm wall 14 and conform approximate the aneurysm neck 16. The aneurysm 10 being treated can have a diameter that is less than the outer diameter of the tubular braid 110 in the predetermined shape so that the braid 110 tends to expand outwardly, providing a force against the aneurysm wall 14 and sealing approximate the perimeter of the aneurysm neck 16.

As illustrated in FIG. 2D, the braid 110 can form the proximal inversion 122a defining the first segment 142a as the braid 110 is further pushed out of the microcatheter 600. The proximal inversion 122a can be positioned approximate the aneurysm neck 16. The distal inversion 124a defining the second segment 144a can also begin to form as the braid 110 is pushed out of the microcatheter 600. As illustrated in FIGS. 2E through 2F, the “S” shape of the second segment 144a can begin to form as the braid 110 is further pushed from the microcatheter 600.

As illustrated in FIG. 2G, once the first portion of the braid 110, which can comprise the first segment 142a, second segment 144a, and third segment 146a, is in place within the aneurysm sac 12, the fourth segment 152a can radially expand outside the aneurysm 10 as the distal portion of the braid 110 continues to exit the microcatheter 600.

As illustrated in FIG. 2H, the fourth segment 152a can then be compressed distally as it continues to radially expand, compressing the fourth segment 152a up into the first portion of the braid 110.

Finally, as illustrated in FIG. 2I, the fourth segment 152a can be compressed distally into the first portion of the braid 110, at least partially occluding the neck 16 of the aneurysm 10 and the neck opening 126. The pinched end 112 and/or the detachment point 150 can remain external to the aneurysm sac once the fourth segment 152a has reached its final expanded and compressed state. The fourth segment 152a when compressed can be compressed to a minimal thickness as to not become an obstruction to the surrounding blood vessels.

Before the implant 100 is released from the delivery system, the implant 100 can be partially or fully retracted into the microcatheter 600 and repositioned.

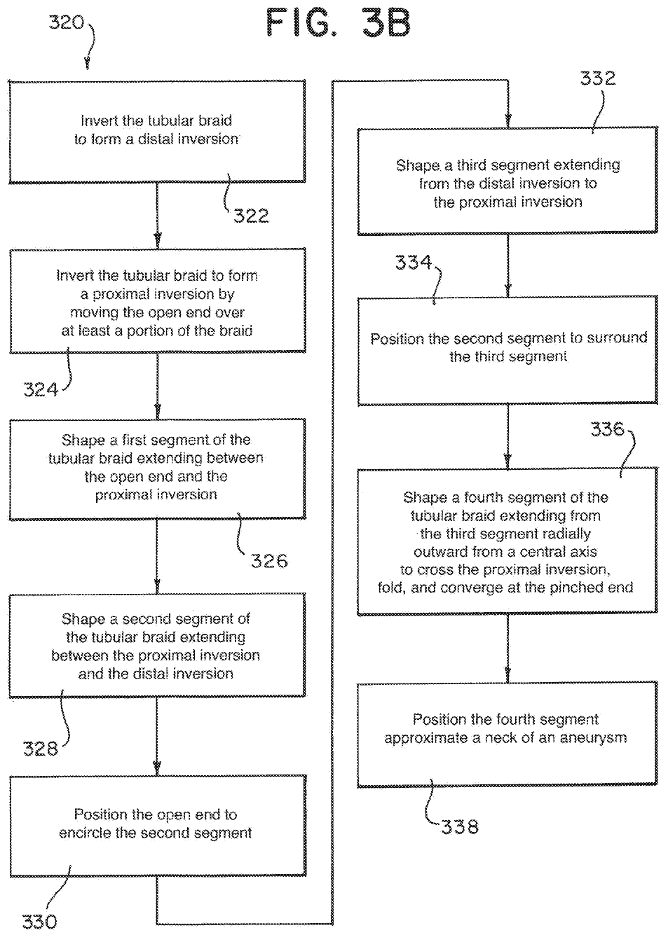

FIG. 3A is a flow diagram for a method 300 for forming an occlusive device to treat an aneurysm 10. Step 310 includes selecting an implant comprising a tubular braid, an open end, and a pinched end. Step 320 includes shaping the tubular braid to a predetermined shape, such as the one illustrated in FIG. 1A. As illustrated in FIG. 3B, step 320 can further comprise additional steps. Step 322 includes inverting the tubular braid to form a distal inversion. Step 324 inverts the tubular braid to form a proximal inversion by moving the open end over at least a portion of the braid. Step 326 includes shaping a first segment of the tubular braid extending between the open end and the proximal inversion. Step 328 shapes a second segment of the tubular braid extending between the proximal inversion and the distal inversion. Step 330 includes positioning the open end to encircle the second segment. Step 332 shapes a third segment extending from the distal inversion to the proximal inversion. Step 334 includes positioning the second segment to surround the third segment. Step 336 shapes a fourth segment of the tubular braid extending from the third segment radially outward from a central axis to cross the proximal inversion, fold inwardly toward the central axis, and converge at the pinched end. Step 338 includes positioning the fourth segment approximate a neck of an aneurysm.

In method 300, step 320 of shaping the tubular braid to the predetermined shape can further include shaping the fourth segment to comprise a diameter greater than or approximately equal to a maximum diameter of the first segment. In method 300, the step 320 of shaping the tubular braid to the predetermined shape can further include shaping the fourth segment to a diameter lesser than a maximum diameter of the first segment. The method 300 can further include shaping the tubular braided implant to a delivery shape sized to traverse a lumen of a microcatheter.

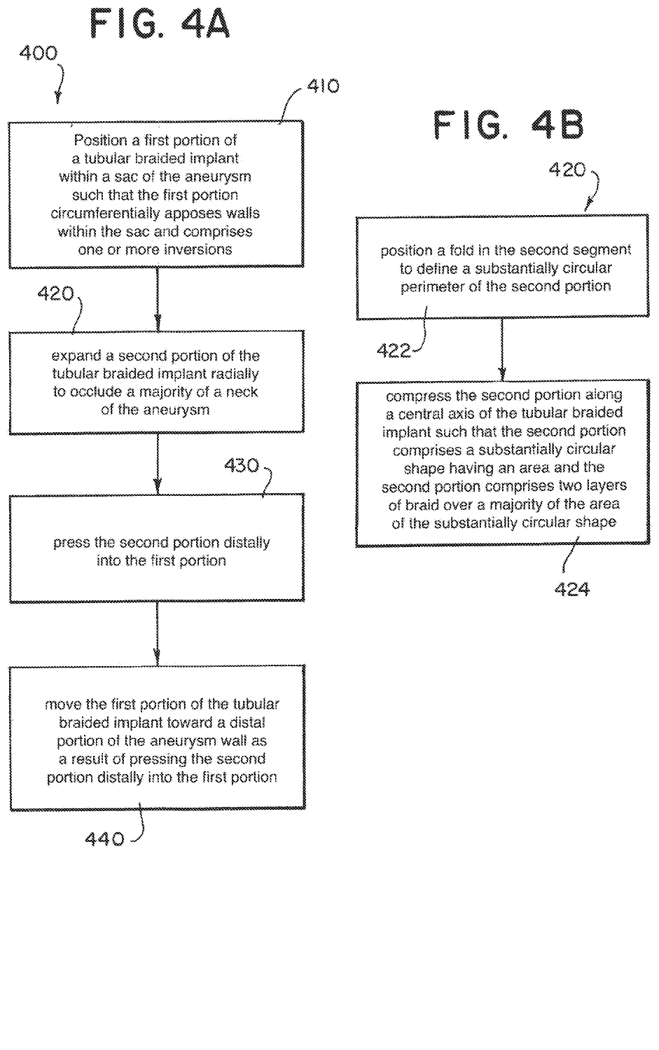

FIG. 4A is a flow diagram for a method 400 for a method for treating an aneurysm 10. Step 410 positions a first portion of a tubular braided implant, the tubular braided implant comprising a tubular braid, an open end, and a pinched end, within a sac of the aneurysm such that the first portion circumferentially apposes walls within the sac. The first portion can include one or more inversions. Step 420 includes expanding a second portion of the tubular braided implant radially to occlude a majority of a neck of the aneurysm. Step 430 presses the second portion distally into the first portion. Pressing the second portion distally into the first portion can create three layers of braid at the neck of the aneurysm. The second portion can cover any spatial gaps between the first portion and the aneurysm neck, and can potentially increase metal coverage, decrease porosity of the implant, and increase stasis and blood flow diversion at the neck of the aneurysm to promote the sealing and healing of the aneurysm. Step 440 includes moving the first portion of the tubular braided implant toward a distal portion of the aneurysm wall as a result of pressing the second portion distally into the first portion.

As illustrated in FIG. 4B, step 420 can further include step 422, which includes positioning a fold in the second segment to define a substantially circular perimeter of the second portion. Step 420 can additionally, or alternatively include step 424, which includes compressing the second portion along a central axis of the tubular braided implant such that the second portion comprises a substantially circular shape having an area and the second portion comprises two layers of braid over a majority of the area of the substantially circular shape.

Step 410 can further include shaping the tubular braided implant to form a columnar post encircling a central axis of the tubular braided implant and extending a majority of a height of the first portion. Step 410 can further include positioning a proximal inversion in the first portion of the tubular braided implant approximate the neck of an aneurysm and positioning a distal inversion in the first portion of the tubular braided implant approximate the distal portion of the aneurysm wall. Step 410 can further include positioning the open end of the tubular braided implant to circumferentially appose the aneurysm wall, shaping a first segment of the tubular braid extending between the open end and the proximal inversion to appose an at least a portion of a wall of the aneurysm within the aneurysm's sac, and shaping a second segment of the tubular braid such that the first segment provides an outwardly radial force in a plane defining a boundary between the aneurysm and blood vessel branches, the force sufficient to appose the first segment to walls of the aneurysm.

Step 430 can further include pressing the second portion of the tubular braided implant against the proximal inversion in the first portion of the tubular braided implant. Step 440 can further include moving the distal inversion in the first portion of the tubular braided implant toward the distal portion of the aneurysm wall.

The method 400 can further include shaping the tubular braided implant to form a columnar post encircling a central axis of the tubular braided implant and extending a majority of a height of the first portion. The method 400 can further include retracting the tubular braid until a desired position is achieved relative to the aneurysm. The method 400 can further comprise shaping the tubular braided implant to a delivery shape sized to traverse a lumen of a microcatheter.

As used herein, the terms “about” or “approximately” for any numerical values or ranges indicate a suitable dimensional tolerance that allows the part or collection of components to function for its intended purpose as described herein.

The descriptions contained herein are examples of embodiments of the invention and are not intended in any way to limit the scope of the invention. The invention contemplates many variations and modifications of the implant, including: alternative delivery methods, alternative braid materials, alternative means for achieving a desired stiffness/flexibility of braid material, additional structures affixed to the implant (e.g. to aid in anchoring the implant, blood flow diversion, embolism formation, etc.), alternative predetermined braid shapes (e.g. one inversion, three inversions, four inversions, five or more inversions, non-radially symmetric shapes, alternative segment shapes, etc.), alternative implanted shapes, etc. The invention contemplates many variations and modifications to constructing the implant to include combinations of the aforementioned variations and modifications of the implant. The invention contemplates many variations and modifications of implanting the implant to accommodate combinations of the aforementioned variations and modifications of the implant. Modifications apparent to one of ordinary skill in the art following the teachings of this disclosure are intended to be within the scope of the claims which follow.

What is claimed is:

1. A method for treating an aneurysm, the method comprising:
positioning a first portion of a tubular braided implant, the tubular braided implant comprising a tubular braid, an open end, and a pinched end, within a sac of the aneurysm such that the first portion circumferentially apposes walls within the sac and comprises one or more inversions;
expanding a second portion of the tubular braided implant radially to occlude a majority of a neck of the aneurysm;
pressing the second portion distally into the first portion; and
moving the first portion of the tubular braided implant toward a distal portion of the aneurysm wall as a result of pressing the second portion distally into the first portion.

2. The method of claim 1, wherein expanding the second portion of the tubular braided implant further comprises:
positioning a fold in the second segment to define a substantially circular perimeter of the second portion; and
compressing the second portion along a central axis of the tubular braided implant such that the second portion comprises a substantially circular shape having an area and the second portion comprises two layers of braid over a majority of the area of the substantially circular shape.

3. The method of claim 1, wherein positioning the first portion of the tubular braided implant further comprises:
shaping the tubular braided implant to form a columnar post encircling a central axis of the tubular braided implant and extending a majority of a height of the first portion.

4. The method of claim 1 further comprising retracting the tubular braid until a desired position is achieved relative to the aneurysm.

5. The method of claim 1 further comprising shaping the tubular braided implant to a delivery shape sized to traverse a lumen of a microcatheter.

6. The method of claim 1, wherein positioning the first portion of the tubular braided implant further comprises:
positioning a proximal inversion in the first portion of the tubular braided implant approximate the neck of an aneurysm; and
positioning a distal inversion in the first portion of the tubular braided implant approximate the distal portion of the aneurysm wall.

7. The method of claim 6, wherein positioning the first portion of the tubular braided implant further comprises:
positioning the open end of the tubular braided implant to circumferentially appose the aneurysm wall;
shaping a first segment of the tubular braid extending between the open end and the proximal inversion to appose an at least a portion of a wall of the aneurysm within the aneurysm's sac;
shaping a second segment of the tubular braid such that the first segment provides an outwardly radial force in a plane defining a boundary between the aneurysm and blood vessel branches, the force sufficient to appose the first segment to walls of the aneurysm.

8. The method of claim 6,
wherein pressing the second portion distally into the first portion further comprises pressing the second portion of the tubular braided implant against the proximal inversion in the first portion of the tubular braided implant; and
wherein moving the first portion of the tubular braided implant toward the distal portion of the aneurysm wall further comprises moving the distal inversion in the first portion of the tubular braided implant toward the distal portion of the aneurysm wall.

9. The method of claim 6, wherein the second portion comprises a diameter greater than or approximately equal to a maximum diameter of the first portion.

10. The method of claim 6, wherein the second portion comprises a diameter lesser than a maximum diameter of the first portion.

11. The method of claim 6,
wherein, when the tubular braid is implanted in an aneurysm, the second portion occludes at least a portion of the aneurysm neck.

12. A method comprising:
selecting an implant comprising a tubular braid, an open end, and a pinched end; and
shaping the tubular braid to a predetermined shape as follows:
inverting the tubular braid to form a distal inversion;
inverting the tubular braid to form a proximal inversion by moving the open end over at least a portion of the braid;
shaping a first segment of the tubular braid extending between the open end and the proximal inversion;
shaping a second segment of the tubular braid extending between the proximal inversion and the distal inversion;
positioning the open end to encircle the second segment;
shaping a third segment extending from the distal inversion to the proximal inversion;
positioning the second segment to surround the third segment;
shaping a fourth segment of the tubular braid extending from the third segment radially outward from a central axis to cross the proximal inversion, folding, and converging at the pinched end; and
positioning the fourth segment approximate a neck of an aneurysm.

13. The method of claim 12, wherein shaping the tubular braid to the predetermined shape further comprises shaping the fourth segment to comprise a diameter greater than or approximately equal to a maximum diameter of the first segment.

14. The method of claim 12, wherein shaping the tubular braid to the predetermined shape further comprises shaping the fourth segment to a diameter lesser than a maximum diameter of the first segment.

15. The method of claim 12,
wherein, when the tubular braid is implanted in an aneurysm, the fourth segment occludes at least a portion of an aneurysm neck.

16. The method of claim 12,
wherein, when the tubular braid is in the predetermined shape, the second segment forms a sack, at least a portion of the third segment is positioned within the sack, and at least a portion of the fourth segment is positioned external to the sack.

17. The method of claim 12, the tubular braid further comprising an implanted shape constrained by an aneurysm, in which the tubular braid comprises:
an outer layer corresponding to the first segment of the predetermined shape and positioned to contact an aneurysm wall of the aneurysm,
a proximal inversion corresponding to the proximal inversion of the predetermined shape and positioned to be placed approximate an aneurysm neck of the aneurysm, and
a sack corresponding to the second segment of the predetermined shape and positioned to appose a portion of the aneurysm wall of the aneurysm and apposing the outer layer.

18. The method of claim 17,
wherein, when the tubular braid is in the implanted shape, the fourth segment comprises a diameter greater than or approximately equal to a maximum diameter of the first segment.

19. The method of claim 17,
wherein, when the tubular braid is in the implanted shape, the fourth segment comprises a diameter lesser than a maximum diameter of the first segment.

20. The method of claim 17,
wherein, when the tubular braid is in the implanted shape, the fourth segment occludes at least a portion of an aneurysm neck.
